SEQUENCE LISTING (8 sequences)

SEQ ID NO: 1 - DNA, 23 bp, Artificial Sequence
  Primer
aactagggaacccactgcttaag

SEQ ID NO: 2 - DNA, 23 bp, Artificial Sequence
  Primer
tctccttctagcctccgctagtc

SEQ ID NO: 3 - DNA, 23 bp, Artificial Sequence
  Primer
tctctagcagtggcgcccgaaca

SEQ ID NO: 4 - DNA, 23 bp, Artificial Sequence
  Primer
tctccttctagcctccgctagtc

SEQ ID NO: 5 - DNA, 19 bp, Artificial Sequence
  Primer
ccccgttctctgggaactc

SEQ ID NO: 6 - DNA, 24 bp, Artificial Sequence
  Primer
tgtatgagaccactctttcccata

SEQ ID NO: 7 - DNA, 22 bp, Artificial Sequence
  Primer
ggtctctctggttagaccagat

SEQ ID NO: 8 - DNA, 22 bp, Artificial Sequence
  Primer
ctgctagagattttccacactg

(19) United States
(12) Patent Application Publication
(10) Pub. No.: US 2015/0031739 A1
(43) Pub. Date: Jan. 29, 2015

(54) PANOBINOSTAT FOR USE IN THE TREATMENT OF HIV-1

(71) Applicants: Aarhus Universitet, Aarhus C (DK); Region Midtjylland, Viborg (DK)

(72) Inventors: Martin Tolstrup, Risskov (DK); Thomas Aagaard Rasmussen, Åbyhøj (DK); Lars Østergaard, Lystrup (DK); Ole Schmeltz Søgaard, Ega (DK); Christel Rothe Brinkmann, Aarhus V (DK)

(73) Assignees: Aarhus Universitet, Aarhus C (DK); Region Midtjylland, Viborg (DK)

(21) Appl. No.: 14/372,706

(22) PCT Filed: Jan. 25, 2013
(86) PCT No.: PCT/DK2013/050025

§ 371 Date: Jul. 16, 2014

Related U.S. Application Data
(60) Provisional application No. 61/591,512, filed on Jan. 27, 2012.

(51) Int. Cl. A61K 31/4045

(52) U.S. Cl. CPC A61K 31/4045
USPC 514/415

(57) ABSTRACT

The present invention relates to uses and methods involving Panobinostat and HIV-1. In particular relates the present invention to Panobinostat for use in the treatment of HIV-1 and especially latent HIV-1. This is done using a low dosage of Panobinostat which is highly effective in depleting the HIV-1 reservoir.

### TECHNICAL FIELD OF THE INVENTION

The present invention relates to uses and methods involving Panobinostat and HIV-1. In particular relates the present invention to Panobinostat for use in the treatment of HIV-1 and especially latent HIV-1.

### BACKGROUND OF THE INVENTION

Despite effective highly active antiretroviral treatment (HAART), HIV-infection persist and rebounds upon treatment interruption, presumably due to latently infected resting CD4+ T-cells.

To achieve eradication of HIV-infection this reservoir of latently HIV-infected cells must be depleted.

Early during HIV infection a latent reservoir is established when newly infected CD4+ T-cells revert to a resting memory state.

These cells harbour integrated proviral DNA capable of resuming HIV-expression upon stimulation, but in the inactive state are unrecognizable to the immune system and unresponsive to antiretroviral drugs.

Histone deacetylase (HDAC) inhibitors are a new class of drugs that have been developed for uses in oncology.

However, these drugs also have the potential to stimulate HIV expression from latently infected resting cells, thereby unmasking the virus and allowing immune clearance and depletion of the viral reservoir.

Highly active antiretroviral therapy (HAART) has markedly reduced HIV-mortality, but HIV-infected patients still experience excess morbidity, partly due to ongoing immune activation and alterations in innate and adaptive immunity, as well as long term side effects of HAART.

In addition, treatment must be maintained lifelong as interruptions result in viral rebound, often within 2-3 weeks, and increased risk of opportunistic disease or death.

Hence, a time-limited treatment capable of eradicating HIV-infection is a desired, but yet unattained goal.

During HAART viral suppression to below the limits of detection of standard assays is frequently achieved, but residual low level viremia that persists for at least 7 years have been demonstrated using ultra sensitive assays.

Whether the residual viremia reflects continuous viral replication in presence of effective HAART or release of virus from stable reservoirs has been the subject of debate in recent years.

Several results indicate that ongoing cycles of replication during suppressive HAART are unlikely, but the cellular sources of the stable reservoir still remain to be fully characterized.

In 1995 integrated HIV DNA was shown to persist within resting CD4+ memory T-cells and in vitro activation led to release of replication competent virus.

These cells appear to be the most significant and certainly the best studied reservoir of HIV infection.

HIV preferentially infects activated CD4+ T-cells that do not survive for more than a few days hereafter, but on rare occasions the virus infects a CD4+ T-cell as it transitions to a resting memory T-cell state.

The result is stably integrated HIV-DNA in a cell with a very long life span capable of resuming viral replication upon subsequent activation.

The molecular mechanisms by which HIV establishes latency are manifold and complex and include enzymatic processes that affect the chromatin organization of the HIV-promoter region, one of the key determinants of transcriptional activity.

Histone deacetylation by HDACs leads to conformational changes that constrict the chromatin and block transcription.

There are 18 known HDACs which are grouped into 4 classes.

Of these, the class I HDACs 1, 2, and 3 may be particular important to maintaining HIV latency.

Accordingly, HDAC inhibitors have consistently shown the ability to reactivate and induce expression of HIV-1 from latently infected cells.

With HIV-1 expression, the infected cells presumably die due to viral cytopathic effects and/or immune mediated killing and there is a progressive reduction in the size of the reservoir, while concurrent HAART prevents spreading of the infection to new cells.

This concept was tested in a clinical study using the weak HDAC inhibitor valproic acid.

Depletion of the latent viral reservoir was seen in 3 of 4 patients, but further studies gave mixed results and the effect could not be sustained.

Newer and far more potent HDAC inhibitors with clinical potential include suberoylanilide hydroxamic acid [SAHA] (Vorinostat), PXD101 (Belinostat), and ITF2357 (Givinostat).

These compounds induce expression of integrated HIV in chronically infected cell lines and ex vivo in resting CD4+ T cells from a viremic HAART treated HIV-patients superior to Valproate.

In addition, ITF2357 decreased surface expression of CXCR4 and CCR5 on CD4+ T-cells and monocytes, respectively.

LBH589 also known as Panobinostat is a novel HDAC inhibitor that exhibits more potent HDAC inhibition than Belinostat, Givinostat and Vorinostat.

In summary, despite considerable recent research in latent HIV-infection in vivo experience is scarce.

HDAC inhibition appears pivotal in targeting HIV latency, but the clinical effect of new and potent HDAC inhibitors on the size of the viral reservoir is unknown.

### SUMMARY OF THE INVENTION

The present invention relates to uses and methods involving Panobinostat and HIV-1.

Thus relates one aspect of the present invention to Panobinostat for use in depleting the latent HIV-1 reservoir.

Another aspect of the present invention relates to Panobinostat for use in the treatment of HIV-1.

An additional aspect of the present invention relates to Panobinostat administered in a dose of 5-60 mg for use in depleting the latent HIV-1 reservoir.

A further aspect of the present invention relates to Panobinostat administered in a dose of 5-60 mg for use in the treatment of HIV-1.

Another aspect of the present invention relates to Panobinostat for use in depleting the latent HIV-1 reservoir, wherein Panobinostat is administered in a phase defined by:

phase (i) a dose of Panobinostat on days 1, 3 and 5 of every other week for 8 weeks, while maintaining background HAART or another antiretroviral therapy as co-therapy.

Yet another aspect of the present invention relates to Panobinostat for use in the treatment of HIV-1, wherein Panobinostat is administered in a phase defined by:

phase (i) a dose of Panobinostat on days 1, 3 and 5 of every other week for 8 weeks, while maintaining background HAART or another antiretroviral therapy as co-therapy.

An additional aspect of the present invention relates to Panobinostat in a dose of 5-60 mg for use in depleting the latent HIV-1 reservoir, wherein Panobinostat is administered in a phase defined by:

phase (i) a dose of Panobinostat on days 1, 3 and 5 of every other week for 8 weeks, while maintaining background HAART or another antiretroviral therapy as co-therapy.

Another aspect of the present invention relates to Panobinostat in a dose of 5-60 mg for use in the treatment of latent HIV-1, wherein Panobinostat is administered in a phase defined by:

phase (i) a dose of Panobinostat on days 1, 3 and 5 of every other week for 8 weeks, while maintaining background HAART or another antiretroviral therapy as co-therapy.

In one embodiment of the present invention comprises the administration and additional phase 0 defined by:

phase 0: a pre-treatment phase of 4 weeks.

In another embodiment of the present invention comprises the administration and additional phase (ii) defined by:

phase (ii): a post-treatment phase of 24 weeks to evaluate the effect of the Panobinostat treatment, where HAART or the other antiretroviral therapy is interrupted the last 8 weeks.

In a further embodiment of the present invention comprises the administration and additional phase (iii) defined by:

phase (iii): optional repeatment of phases 0, (i), and/or (ii).

A further aspect of the present invention relates to Panobinostat formulated for oral administration.

Yet another aspect of the present invention relates to a method for treating HIV-1 comprising administration of a pharmaceutically effective amount of Panobinostat to an individual in need thereof.

Another aspect of the present invention relates to method for depleting the latent HIV-1 reservoir comprising administration of a pharmaceutically effective amount of Panobinostat to an individual in need thereof.

In one embodiment of the present invention is HIV-1 latent HIV-1.

### BRIEF DESCRIPTION OF THE FIGURES

FIG. 1 shows HIV-1 expression in U1 and ACH2 cells treated with histone deacetylase inhibitors.

HIV-1 expression in U1 and ACH2 cells treated with LBH589 (Panobinostat), ITF2357 (Givinostat), PXD101 (Belinostat), SAHA (Vorinostat), and Valproate (VPA) for 48 hours. Mean p24 concentrations of 8 (LBH589 and PMA) and 15 (SAHA, ITF2357, PXD101, and VPA) separate experiments in each cell line are depicted in graph.

FIG. 2 shows direct comparison of HIV-1 expression with indicated concentration of each histone deacetylase inhibitor.

HIV-1 expression with indicated concentrations of histone deacetylase inhibitors. Mean concentration of p24 is depicted; error bars show SEM.

FIG. 3 shows fold increase in p24 levels compared to untreated cultures at clinically achievable concentrations.

Fold increase in p24 levels compared to untreated cultures with varying concentration of PXD101, ITF2357, SAHA, and VPA. Two different concentrations within the clinically achievable range of each histone deacetylase inhibitor are chosen as illustrated.

FIG. 4 shows CD69 expression in CD4+ T-cells treated with histone deacetylase inhibitors.

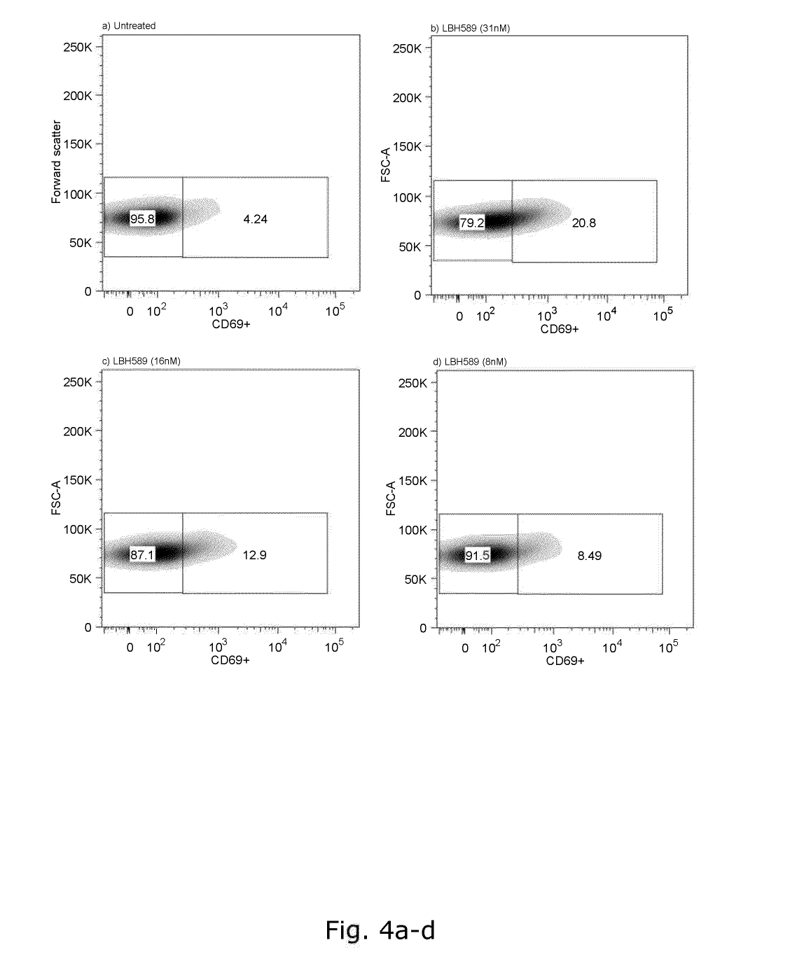

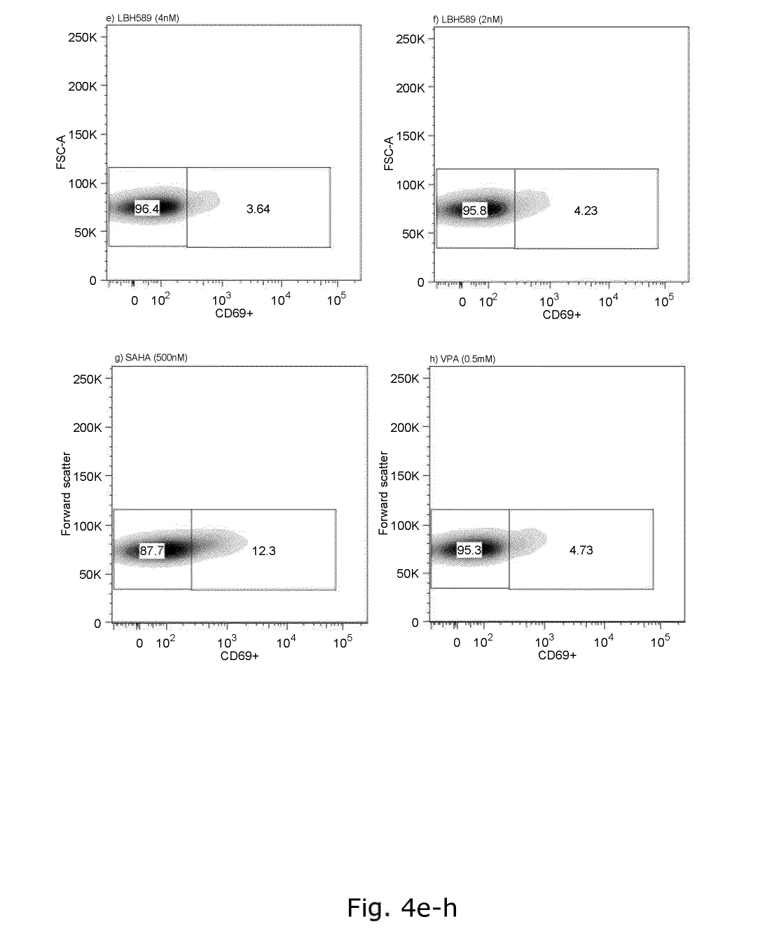

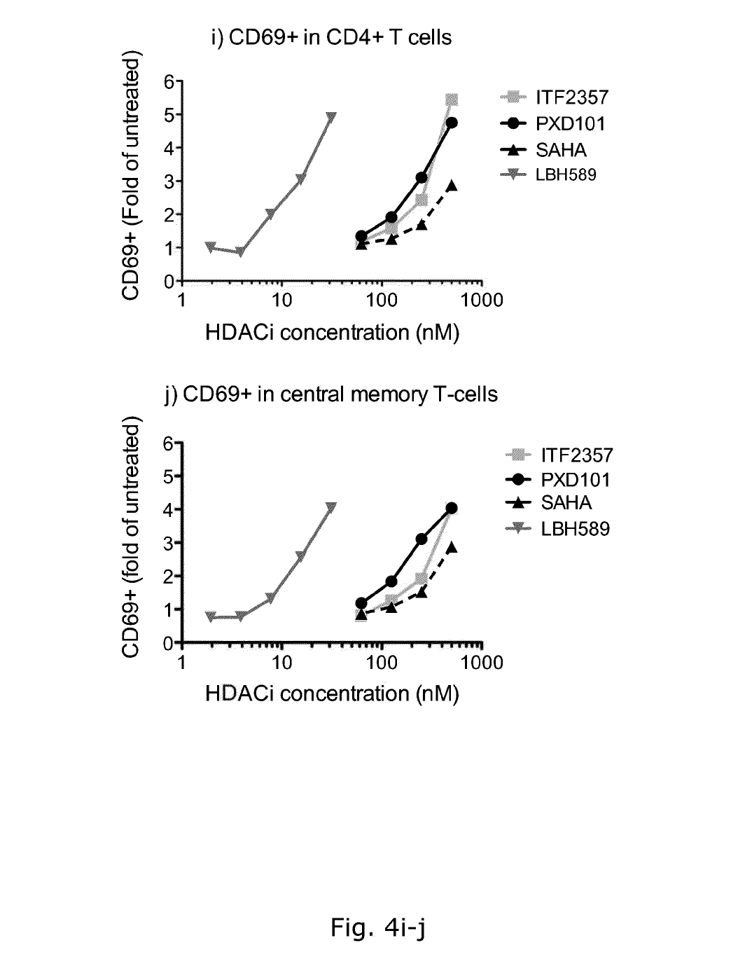

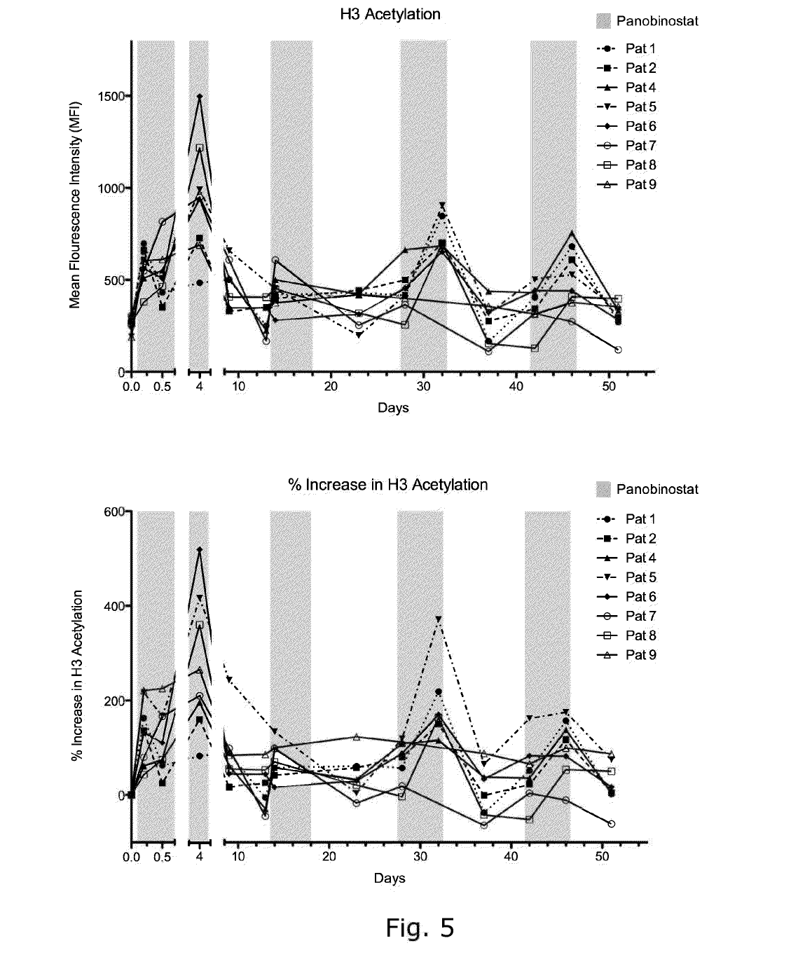

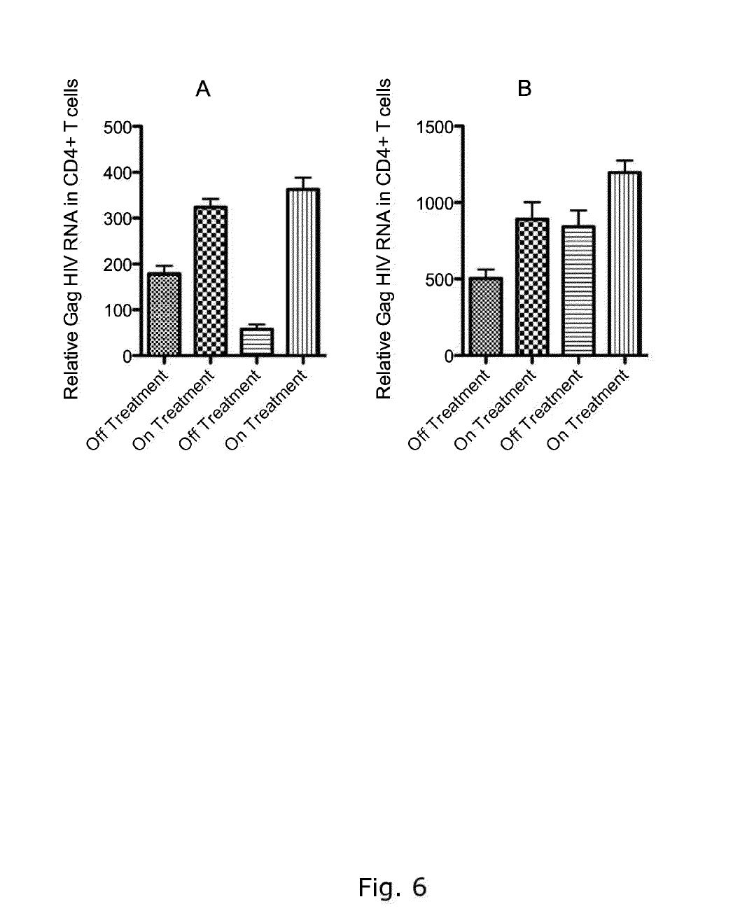

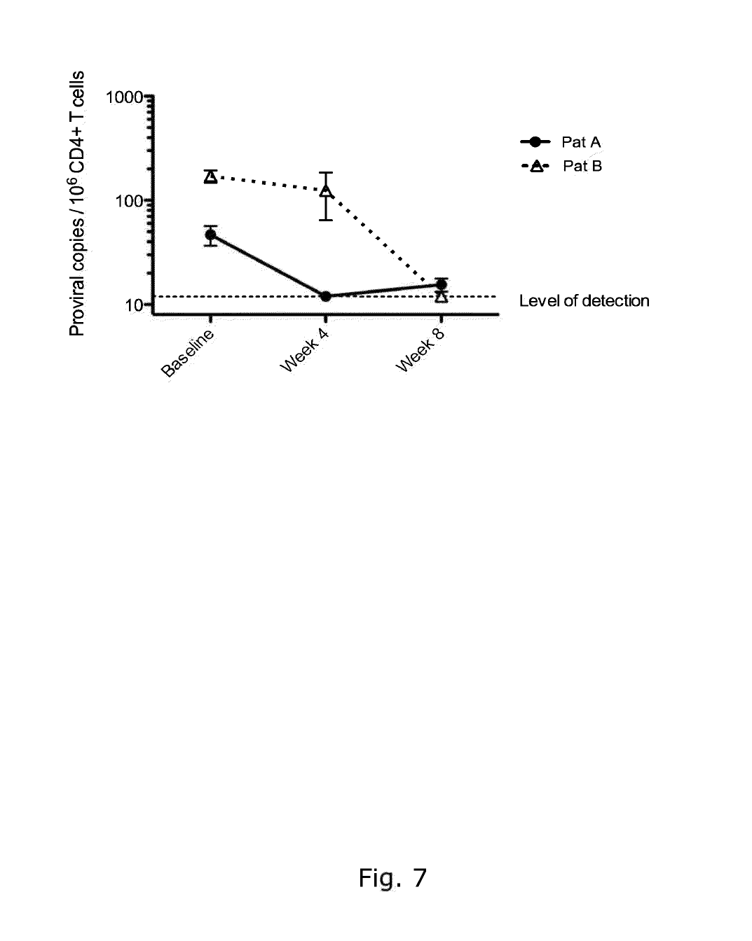

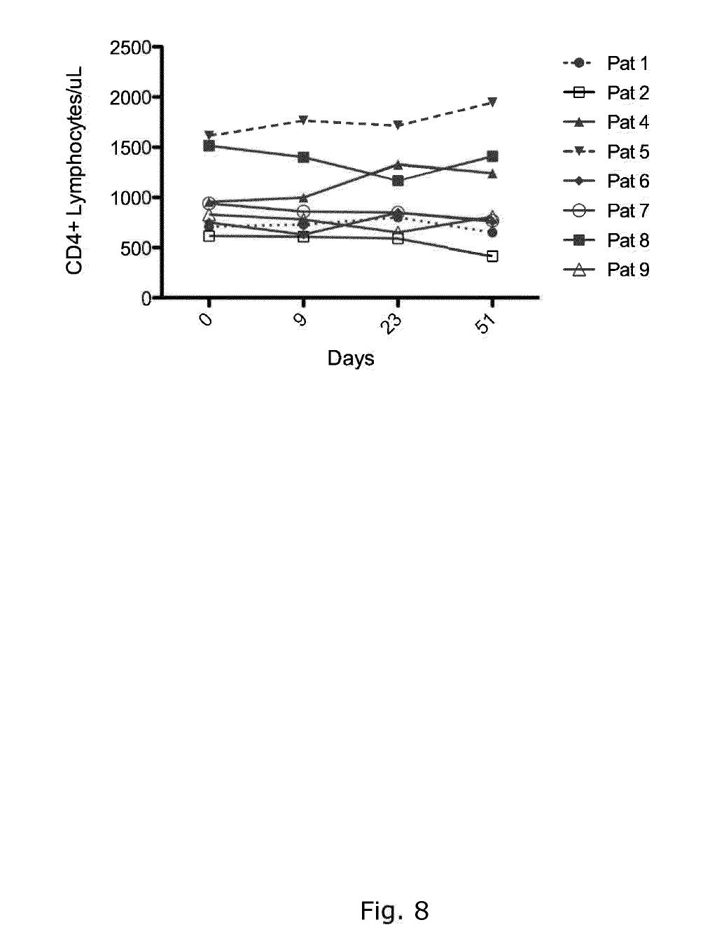

PBMCs from healthy donors were treated 16 hours with ITF2357, PXD101, SAHA (all 62.5-500 nM), LBH589 (2-31 nM), and Valproate (0.5 mM). Expression of the early activation marker CD69 was assessed by flow cytometry in 8 separate experiments with data from one representative experiment shown in figure. Density plots of CD69 expression in CD4+ T-cells treated with LBH589 is illustrated in FIG. 4a-f; similar plots of CD69 expression in CD4+ T-cells treated with SAHA (500 nM) and VPA (0.5 mM) are shown in FIG. 4g-h for comparison. CD69 expression in CD4+ T-cells and central memory T-cells (CD45RA−, CD27+, CCR7+; T_{cm}) across the full range of concentrations used for ITF2357, PXD101, SAHA, and LBH589 (2-500 nM) are shown in FIG. 4i-j.

FIG. 5 shows the histone H3 acetylation increase for each patient

On day 4, there was an average increase of 267% from baseline. In the off-treatment weeks, levels of histone acetylation dropped to near baseline levels.

FIG. 6 shows the effect of cyclic panobinostat dosing on the levels of cell-associated unspliced HIV RNA was evident

In patient A, the levels increased from 178 to 323 and from 57 to 362 Gag HIV RNA copies in the two off/on treatment cycles analyzed. For patient B, the levels were generally higher but a similar increase was observed with off/on treatment status from 504 to 841 and from 892 to 1196 Gag HIV RNA copies in the two cycles analyzed.

FIG. 7 shows the determined total proviral DNA content in purified CD4+ T cell in two HIV-1 patients over the course of cyclic panobinostat administration in vivo The observed drop to below levels of detection in the PCR assay appears to be of major significance with regard to the utility of panobinostat as a prime candidate for HIV-1 eradication purposes.

FIG. 8 shows the effect of intermittent panobinostat dosing over 51 days on CD4+ T cell counts in blood

The TIW oral dosing of 20 mg every other week does not lead to depletion of CD4 T cells nor does it give rise to safety concerns supporting that panobinostat can be safely administered to HIV-infected adults on HAART.

The present invention will now be described in more detail in the following.

### DETAILED DESCRIPTION OF THE INVENTION

The present inventors have investigated the potential of Panobinostat for eradicating the latent HIV-1 reservoir and have surprisingly found that a low dose of Panobinostat is very effective for this purpose.

The rationale for the reduced dose is based on studies in latently infected cell lines (U1 and ACH2), where HIV-1 expression is induced in the 8-32 nM concentration range.

Examples 1-6 support that Panobinostat surprisingly is able to significantly lower the proviral DNA (example 5) and has the ability to reactivate HIV-1 in vivo as determined by levels of unspliced HIV Gag RNA in CD4+ T cells (example 4) while no depletion of CD4+ T cells is seen (example 6).

This means that treatment of HIV infected individuals with Panobinostat does not give rise to safety concerns.

Uses

Accordingly relates an object of the present invention to Panobinostat for use in depleting the HIV-1 reservoir.

In one embodiment is this HIV-1 reservoir the latent HIV-1 reservoir as described below.

Another object of the present invention relates to Panobinostat for use in the treatment of HIV-1.

Such treatment could be through purging or depletion of the HIV-1 reservoir, but also through anti-inflammatory effects that could be beneficial in the treatment of HIV-1.

Panobinostat

Panobinostat also known as LBH-589 is a HDAC inhibitor belonging to a structurally novel cinnamic hydroxamic acid class of compounds.

Panobinostat was developed by Novartis for the treatment of various cancers and has the chemical formula: N-Hydroxy-3-[4-[2-(2-methyl-1H-indol-3-yl)ethylaminomethyl]phenyl]-2(E)-propenamide.

It is a highly potent class I/II pan-HDAC inhibitor that has shown anti-latency activity in pre-clinical models.

Histone deacetylases target lysine groups on chromatin and transcription factors and various non-histone proteins such as p53, tubulin, heat shock protein 90 (HSP90) and retinoblastoma protein (Rb).

Panobinostat is formulated as an oral capsule and as a solution for intravenous (i.v.) injection.

HIV

Human immunodeficiency virus (HIV) is a lentivirus (a member of the retrovirus family) that causes acquired immunodeficiency syndrome (AIDS).

HIV infects vital cells in the human immune system such as helper T cells (specifically CD4+ T cells), macrophages, and dendritic cells.

HIV infection leads to low levels of CD4+ T cells through three main mechanisms: First, direct viral killing of infected cells; second, increased rates of apoptosis in infected cells; and third, killing of infected CD4+ T cells by CD8 cytotoxic lymphocytes that recognize infected cells.

When CD4+ T cell numbers decline below a critical level, cell-mediated immunity is lost, and the body becomes progressively more susceptible to opportunistic infections.

HIV-1 is the most common and pathogenic strain of the virus.

The total amount of viral particles constitutes the HIV-1 reservoir.

Latent HIV

Virus latency (or viral latency) is the ability of a pathogenic virus to lay dormant (latent) within a cell, denoted as the lysogenic part of the viral life cycle.

A latent infection is a phase in certain viruses' life cycles in which after initial infection, virus production ceases.

However, the virus genome is not fully eradicated.

The result of this is that the virus can reactivate and begin producing large amounts of viral progeny without the host being infected by new exogenousvirus.

In the field of HIV research, proviral latency in specific long-lived cell types is the basis for the concept of one or more viral reservoirs, referring to locations (cell types or tissues) characterized by persistence of latent virus.

Specifically, the presence of replication-competent HIV in resting CD4-positive T cells, allows the virus to persist for years despite prolonged exposure to antiretroviral drugs.

This latent reservoir of HIV may explain the inability of antiretroviral treatment (HAART) to cure HIV infection.

Thus, in one embodiment of the present invention is HIV-1 a latent HIV-1 infection.

This remaining HIV-1 infection constitutes the latent HIV-1 reservoir.

HAART

Antiretroviral drugs are medications for the treatment of infection by retroviruses, primarily HIV.

When several such drugs, typically three or four, are taken in combination, the approach is known as Highly Active Antiretroviral Therapy, or HAART.

There are different classes of antiretroviral drugs that act on different stages of the HIV life-cycle.

Antiretroviral (ARV) drugs are broadly classified by the phase of the retrovirus life-cycle that the drug inhibits.

Entry inhibitors (or fusion inhibitors) interfere with binding, fusion and entry of HIV-1 to the host cell by blocking one of several targets. Maraviroc and enfuvirtide are the two currently available agents in this class.

CCR5 receptor antagonists are the first antiretroviral drugs which do not target the virus directly. Instead, they bind to the CCR5 receptor on the surface of the T-Cell and block viral attachment to the cell. Most strains of HIV attach to T-Cells using the CCR5 receptor. If HIV cannot attach to the cell, it cannot gain entry to replicate.

Nucleotide reverse transcriptase inhibitors (NRTI) inhibit reverse transcription by being incorporated into the newly synthesized viral DNA strand as a faulty nucleotide. This causes a chemical reaction resulting in DNA chain termination.

Non-Nucleoside reverse transcriptase inhibitors (NNRTI) inhibit reverse transcriptase directly by binding to the enzymes polymerase site and interfering with its function.

Protease inhibitors (PIs) target viral assembly by inhibiting the activity of protease, an enzyme used by HIV to cleave nascent proteins for the final assembly of new virions.

Integrase inhibitors (IIs) inhibit the enzyme integrase, which is responsible for integration of viral DNA into the DNA of the infected cell. There are several integrase inhibitors currently under clinical trial, and raltegravir became the first to receive FDA approval in October 2007.

Maturation inhibitors inhibit the last step in gag processing in which the viral capsid polyprotein is cleaved, thereby blocking the conversion of the polyprotein into the mature capsid protein (p24). Because these viral particles have a defective core, the virions released consist mainly of non-infectious particles. Alpha interferon is a currently available agent in this class. Two additional inhibitors under investigation are bevirimat and Vivecon.

Antiretroviral combination therapy defends against resistance by suppressing HIV replication as much as possible.

Combinations of antiretrovirals create multiple obstacles to HIV replication to keep the number of offspring low and reduce the possibility of superior mutations.

If a mutation that conveys resistance to one of the drugs being taken arises, the other drugs continue to suppress reproduction of that mutation.

With rare exceptions, no individual antiretroviral drug has been demonstrated to suppress an HIV infection for long; these agents must be taken in combinations in order to have a lasting effect.

As a result, the standard of care is to use combinations of antiretroviral drugs. Combinations usually comprise two nucleoside-analogue RTIs (NRTI) and one non-nucleoside-analogue RTI (NNRTI) or protease inhibitor (PI).

This three drug combination is commonly known as a triple cocktail.

Most current HAART regimens consist of three drugs: 2 NRTIs+a PI/NNRTI/II.

Thus in one embodiment of the present invention comprises HAART Panobinostatat least one antiretroviral drug selected from the group consisting of a NRTI, a NNRTI, a PI, a II and a maturation inhibitor.

In one embodiment of the present invention comprises HAART at least four other antiretroviral drugs, such as three, such as two.

In yet another embodiment of the present invention is HIV-1 treated with an alternative antiretroviral therapy than HAART.

Depletion and Treatment

Therapy or treatment is the attempted remediation of a health problem, usually following a diagnosis.

In the medical field, therapy is synonymous with the word “treatment”.

A treatment treats a problem, and may lead to its cure, but treatments often ameliorate a problem only for as long as the treatment is continued, especially in chronic diseases.

In the present case there is no cure for AIDS, but treatments are available to slow down the harm done by HIV and delay the fatality of the disease.

Also in the present case is HAART a treatment for HIV-1 that does not cure the viral infection, but instead limit the HIV-1 infection to being a latent viral infection i.e. a persistent viral infection where virus production ceases or at least is diminished, but the virus genome is not fully eradicated.

The remaining latent reservoir of HIV is the likely explanation of the inability of antiretroviral treatment (HAART) to cure HIV infection.

The present invention presents Panobinostat as a way of purging or depleting this latent reservoir.

This depletion results in a significantly lower proviral load in the treated individual.

In the present context refers treatment or depletion of HIV-1 or the latent reservoir of HIV-1 to a change in copies of total proviral HIV-DNA per 10⁶ CD4+ T-cells.

Alternative ways of defining treatment or depletion of HIV-1 or the latent reservoir of HIV-1 includes:
Though a change from pre-treatment phase to post-treatment phase in the size of the latent HIV-reservoir, as measured by copies of integrated proviral HIV-DNA per 10⁶ CD4+ T-cells.
Through a change from pre-treatment phase to post-treatment in the size of the latent HIV-reservoir, as measured by copies of total proviral HIV-DNA per 10⁶ resting CD4+ T-cells or a subject with undetectable proviral HIV-DNA per 10⁶ resting CD4+ T-cells after treatment.
Through a change from baseline in unspliced HIV-RNA in CD4+ T-cells during study treatment.

In one embodiment of the present invention is the effect of Panobinostat enhanced in patients that have a low total HIV proviral DNA content (copies/10⁶ CD4+ cells) before treatment.

Thus in one embodiment of the present invention is the total HIV DNA content before treatment with Panobinostat less that 5000×10⁶, such as less than 1000×10⁶, such as less than 500×10⁶, such as less than 200×10⁶, such as less than 100×10⁶.

In a preferred embodiment of the present invention is the total HIV DNA content before treatment with Panobinostat less that 500×10⁶.

In another embodiment of the present invention is the Gag HIV RNA copies significantly lowered by the Panobinostat treatment described herein.

The retroviral activation of latent HIV-1 with Panobinostat described herein can be supplemented by activation of the immune system.

Such activation of the immune system can be done by for example PEGylated interferon.

Other examples of activators of the immune system or immune boosters are Toll-like receptor (TLR) agonist, Complete Freund's Adjuvant (CFA), antibodies targeting the programmed death 1 (PD-1) receptor, recombinant interleukin-7 (IL-7) or IL-15.

Thus relates one embodiment of the present invention to the use of a combination of retroviral activation with Panobinostat with an immune booster to deplete the latent HIV-1 reservoir.

Statistics

Change is measured based on experimental data from the subjects undergoing treatment.

A change can be either two single values that are compared directly or two or more data sets that are compared using statistical analysis.

There are many ways of making statistical comparisons of data sets.

Examples include the p-value which is the probability of obtaining a test statistic at least as extreme as the one that was actually observed, assuming that the null hypothesis is true.

In statistics, a result is called “statistically significant” if it is unlikely to have occurred by chance.

The significance level is usually denoted by the Greek symbol a (lowercase alpha).

Popular levels of significance are 10% (0.1), 5% (0.05), 1% (0.01), 0.5% (0.005), and 0.1% (0.001).

If tests of significance gives a p-value lower than the significance level a, the null hypothesis is rejected.

Such results are informally referred to as ‘statistically significant’.

For example, if someone argues that “there's only one chance in a thousand this could have happened by coincidence,” a 0.001 level of statistical significance is being implied.

The lower the significance level, the stronger the evidence required.

Choosing level of significance is a somewhat arbitrary task, but for many applications, a level of 5% is chosen, for no better reason than that it is conventional.

Thus in one embodiment of the present invention is the change as described above with a significance level of 50%, such as 10%, such as 5%, such as 1%, such as 0.1%, such as 0.01%, such as 0.001%, such as 0.0001%.

Dosage

The present inventors have found that a surprisingly low concentration of Panobinostat can be used for the treatment of HIV-1.

Thus relates an aspect of the present invention to Panobinostat administered in a dose of 5-60 mg for use in depleting the latent HIV-1 reservoir.

Another aspect of the present invention relates to Panobinostat administered in a dose of 5-60 mg for use in the treatment of HIV-1.

In an embodiment of the present invention is Panobinostat administered in a dose of 5-50 mg.

In an embodiment of the present invention is Panobinostat administered in a dose of 10-35 mg.

In another embodiment of the present invention is Panobinostat is administered in a dose of 15-30 mg.

In yet another embodiment of the present invention is Panobinostat administered in a dose of 20 mg.

The dosage can also be in a very low dose of 5-10 mg.

Intermittent treatment with plasma concentrations well above the threshold for inducing HIV-1 expression is believed to provide the most optimal balance between effect and side effects.

As an example is the maximal plasma concentration achieved with 20 mg oral Panobinostat is approximately 60 nM.

Thus, in an embodiment of the present invention is the effective plasma concentration of Panobinostat 5-200 nM, such as 10-100 nM, such as 20-80 nM, such as 30-70 nM, such as 40-60 nM.

In a preferred embodiment of the present invention is the resulting plasma concentration of Panobinostat 50-70 nM.

The optimal dose depends on several factors including sex and weight.

The dose that is effective is also known as the pharmaceutically effective dose.

Administration Pattern

The objective of drug therapy is to obtain a certain effective plasma concentration within the therapeutic window that allows treatment of the target disease.

The dosage regimen is the modality of drug administration that is chosen to reach the therapeutic objective.

This depends on the drug used, the condition to be treated, and the patient's characteristics.

The decisions defining dosage regimen are about: route of administration, galenic formulation, unit dose, frequency, loading dose, and length of treatment.

The present inventors have found that the administration pattern or dosage regimen is important in order to achieve the optimal treatment of a subject (patient).

Thus relates an aspect of the present invention to Panobinostat for use in depleting the latent HIV-1 reservoir, wherein Panobinostat is administered in a phase (i) defined by:

phase (i) a dose of Panobinostat on days 1, 3 and 5 of every other week for 8 weeks, while maintaining background HAART as co-therapy.

Another aspect of the present invention relates to Panobinostat for use in the treatment of HIV-1, wherein Panobinostat is administered in a phase defined by:

phase (i) a dose of Panobinostat on days 1, 3 and 5 of every other week for 8 weeks, while maintaining background HAART as co-therapy.

In one embodiment of the present invention comprises phase (i) a dose of Panobinostat on days 2, 4 and 6 or days 3, 5 and 7.

In another embodiment of the present invention is Panobinostat given every day of a week, such as every second day or every third day.

In yet another embodiment of the present invention is the dose given every fourth week, such as every third week, such as every second week, such as every week.

In a further embodiment of the present invention is Panobinostat given for 24 weeks, such as 18 weeks, such as 16 weeks, such as 14 weeks, such as 12 weeks, such as 10 weeks, such as 8 weeks, such as 6 weeks, such as 4 weeks, such as 2 weeks.

Both of the above aspects of administration patterns can comprise one or more further phases that can be used to optimize the treatment.

Thus relates one embodiment of the present invention to an administration pattern further comprising a phase 0 before phase (i) defined by:

phase 0: a pre-treatment phase of 4 weeks.

The pre-treatment phase 0 is a phase where the subject (patient) to be treated is monitored and evaluated.

In one embodiment of the present invention is the pre-treatment phase 0 2-5 years, such as less than 1 year, such as 10 months, such as 6 months, such as 3 months, such as 6 weeks, such as 4 weeks, such as 2 weeks, such as 1 week.

In another embodiment of the present invention comprises the administration pattern also a further phase (ii) defined by:

phase (ii): a post-treatment phase of 24 weeks to evaluate the effect of the Panobinostat treatment, where HAART is interrupted the last 8 weeks.

The post-treatment phase (ii) is used to evaluate the effect of the treatment on the patient.

The length of the post-treatment phase (ii) is 2-5 years, such as less than one year, such as 10 months, such as 24 weeks, such as 20 weeks, such as 16 weeks, such as 10 weeks, such as 6 weeks.

The post-treatment phase (ii) includes interruption of HAART in order to evaluate the effect of Panobinostat on the latent HIV-1 reservoir.

Ideally would the length of the interruption be forever indicating that the patient is cured completely.

Alternatively is HAART interrupted in order to evaluate when HIV-1 can be detected in the patient. The interruption can therefore be 2-5 years, such as less than one year, such as 10 months, such as 24 weeks, such as 20 weeks, such as 16 weeks, such as 10 weeks, such as 6 weeks, such as 4 weeks, such as 2 weeks, such as 1 week.

In yet another embodiment of the present invention comprises the administration pattern a phase (iii) defined by:

phase (iii): optional repeatment of one or more of phases 0, (i), and/or (ii).

Phase (iii) is a phase where one or more of the above phase can be repeated in order to optimize the treatment to the patient's needs.

Phase (iii) can be repeated up to five times, such as four times, such as three times, such as two times, such as one time.

For most drugs, a usual dosage regimen is proposed by the manufacturer and approved by registration authorities.

Such regimen should suit the patient's needs.

As a principle, individualization of the dosing regimen should be considered systematically, leading in selected cases to apply unusual dosing decisions in order to tailor the treatment to a patient condition.

For example, the standard regimen must be adapted to the patient's characteristics (e.g. weight and age) and to the presence of co-morbidity that affects the drug's pharmacokinetics (e.g. renal failure, liver disease).

The administration pattern or dosage regimen can therefore also be adjusted depending on factors like sex, age, co-mobidity, route of administration, galenic formulation, unit dose, frequency and loading dose.

Dosage and Administration Pattern

As mentioned above is the dosage regimen very important and could ideally be tailored to every single patient's needs.

Thus relates one aspect of the present invention to Panobinostat in a dose of 5-50 mg for use in depleting the latent HIV-1 reservoir, wherein Panobinostat is administered in a phase defined by:

phase (i) a dose of Panobinostat on days 1, 3 and 5 of every other week for 8 weeks, while maintaining background HAART as co-therapy.

Another aspect of the present invention relates to Panobinostat in a dose of 5-50 mg for use in the treatment of latent HIV-1, wherein Panobinostat is administered in a phase defined by:

phase (i) a dose of Panobinostat on days 1, 3 and 5 of every other week for 8 weeks, while maintaining background HAART as co-therapy.

One embodiment of the present invention relates to an administration pattern further comprising a phase 0 before phase (i) defined by:

phase 0: a pre-treatment phase of 4 weeks.

In another embodiment of the present invention comprises the administration pattern also a further phase (ii) defined by:

phase (ii): a post-treatment phase of 24 weeks to evaluate the effect of the Panobinostat treatment, where HAART is interrupted the last 8 weeks.

In yet another embodiment of the present invention comprises the administration pattern a phase (iii) defined by:

phase (iii): optional repeatment of one or more of phases 0, (i), and/or (ii).

The design of the optimal doses and length of the individual phase is described in the above sections.

Product

Panobinostat is administered to patient in very low doses.

Thus relates one aspect of the present invention to Panobinostat (LBH-589) formulated in a 5-60 mg dose, such as 5-50 mg dose, such as a 10-30 mg dose, such as a 20 mg dose, such as a 10 mg dose, such as a 5 mg dose.

Routes of Administration

Panobinostat can be administered using several different routes of administration.

Thus relates of aspect of the present invention to Panobinostat formulated for oral administration or i.v. injection.

In such aspects could Panobinostat be formulated with at least one immunologically or pharmaceutically acceptable carrier, adjuvant, excipient or diluent.

Method of Treatment

The present inventors have also presented a method of treating HIV-1.

Thus relates one aspect of the present invention to a method for treating HIV-1 comprising administration of a pharmaceutically effective amount of Panobinostat to an individual in need thereof.

Another aspect of the present invention relates to a method for depleting the latent HIV-1 reservoir comprising administration of a pharmaceutically effective amount of Panobinostat to an individual in need thereof.

### Examples

### Example 1

### Comparison of Various HDACi for Stimulating HIV-1 Expression from Latently Infected Cell Lines

Materials and Methods

HDACi stock: Belinostat, Givinostat, Vorinostat and Panobinostat are stored in stock solutions of 2 mM in DMSO.

HIV-1 activation from U1 cells: U1 cells (obtained through the NIH AIDS Reagent Program) are seeded at 2*10⁵/well in a 96-well format in 100 uL RPMI media supplemented with 10% heat inactivated Foetal Bovine Serum and 1% penicillin/streptomycin. HDACi and PMA as positive control is added at desired concentrations in 100 uL. Identical DMSO levels is used for untreated control. All stimulations are performed in triplicate. 48 hours later supernatant is harvested and left for inactivation in 1% Empegen for 1 hour. Levels of HIV-1 p24 gag levels is determined by ELISA as described by the manufacturer (Aalto Bioreagents, Dublin, Ireland).

HIV-1 activation from ACH2 cells: ACH2 cells (obtained through the NIH AIDS Reagent Program) are seeded at 1*10⁵/well in a 96-well format in 100 uL RPMI media supplemented with 10% heat inactivated Foetal Bovine Serum and 1% penicillin/streptomycin. HDACi and PMA as positive control is added at desired concentrations in 100 uL. Identical DMSO levels is used for untreated control. All stimulations are performed in triplicate. 48 hours later supernatant is harvested and left for inactivation in 1% Empegen for 1 hour. Samples are diluted 1/500 and levels of HIV-1 p24 gag is determined by ELISA as described by the manufacturer (Aalto Bioreagents, Dublin, Ireland).

Activation of primary T lymphocytes: PBMC's are purified using Ficoll Paque gradient centrifugation from fresh blood obtained from healthy donors. 5*10⁵ cells are stimulated by different concentrations of HDACi in final volume of 500 uL RPMI media supplemented with 10% heat inactivated Foetal Bovine Serum and 1% penicillin/streptomycin. After 16 hours cells are harvested and washed once in PBS. Cells are stained with Live-dead near IR at a final concentration of 2 uM (Invitrogen, Denmark) for 30 min on ice. Cells are washed twice in PBS. Five uL Fc Block (Sigma, Denmark) at 10 mg/mL is added and cells are incubated 10 min on ice. Cells are subsequently stained with 5 uL CD4 PerCPCy5.5, 5 uL CD27 PE-Cy7, 7 uL CD45RA FITC, 20 uL CCR7 PE all (BD, USA) and 10 uL CD69 APC (BioLegend, USA) for 30 min on ice. Lastly, cells are washed once in PBS and resuspended in FACS flow buffer and stored on ice until analysis by flow cytometry.

Results

The various HDACi displayed significant differences in potency when stimulating HIV-1 expression from the latently infected cell lines with LBH589>ITF2357 and PXD101>SAHA>VPA.

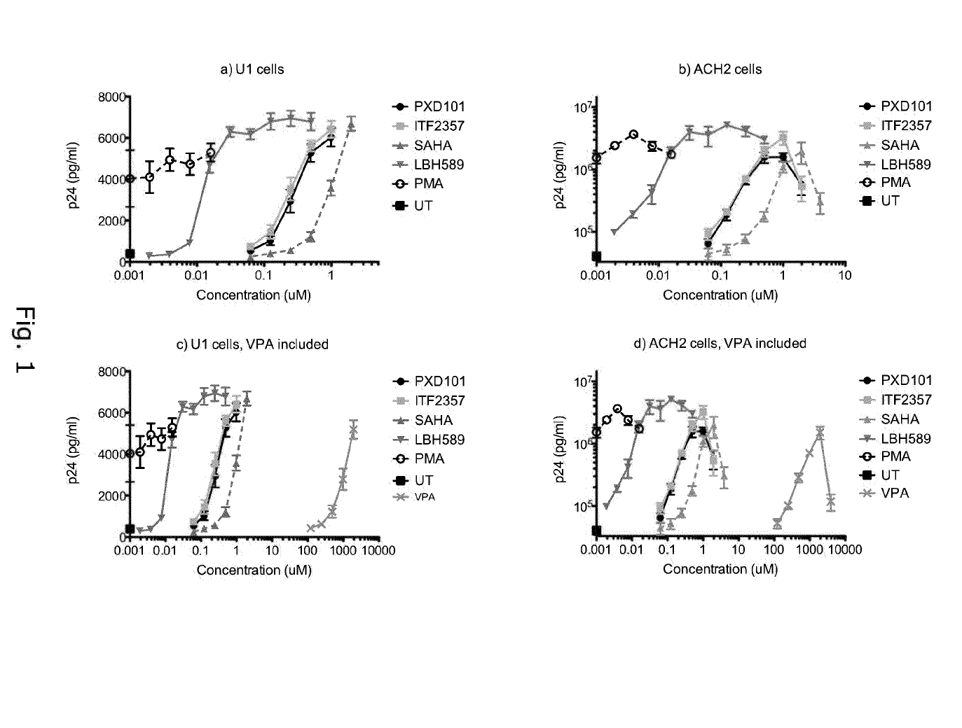

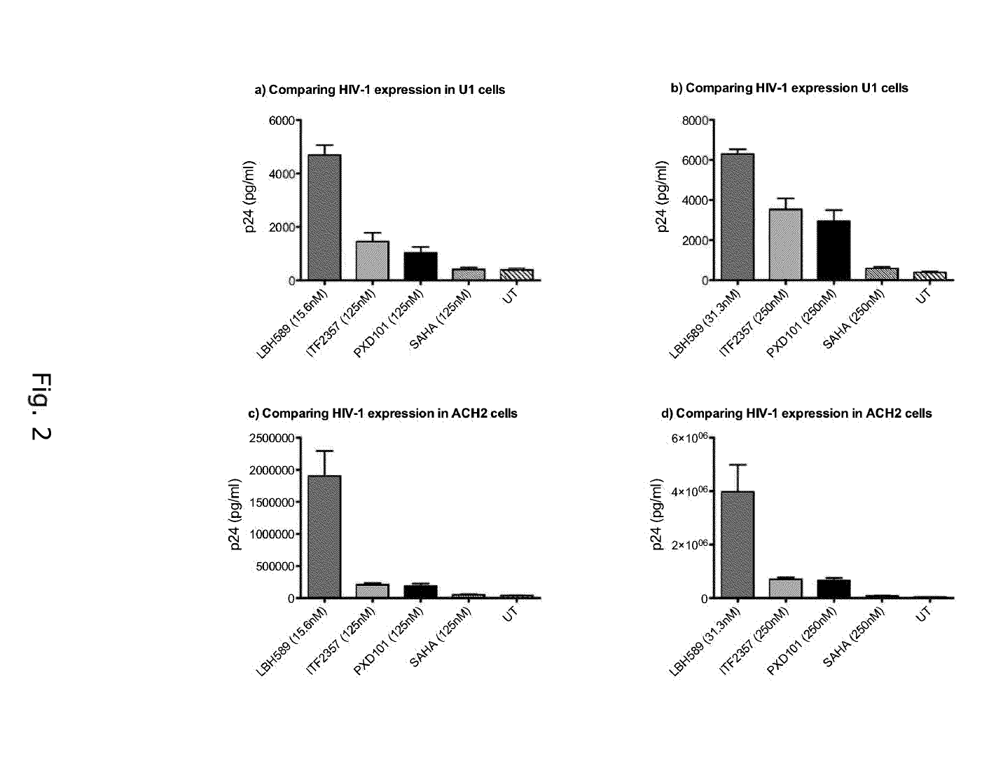

LBH589 was significantly more potent than all other HDACi and showed great potential for reversing latency even in the very low concentration range (FIG. 1).

Direct comparisons of the HDACi at the concentration obtained with clinical use showed that LBH589 induces HIV-1 expression from the latently infected cell lines to much higher levels with LBH589>ITF2357 and PXD101>SAHA (FIG. 2).

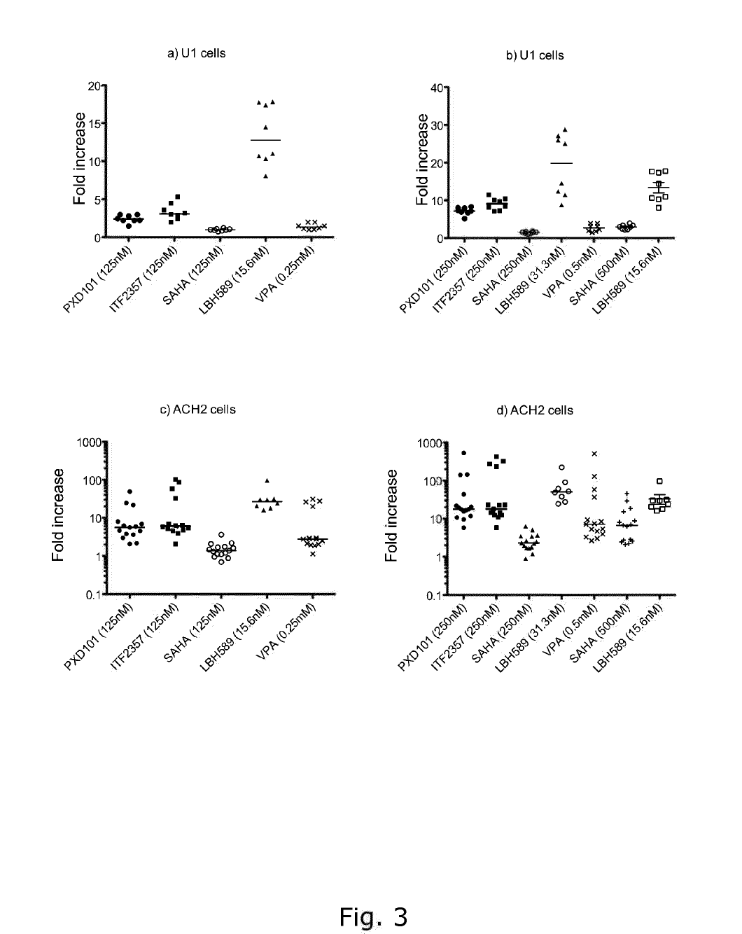

In U1 cells, with reference to untreated cultures, LBH589 increased median p24 levels 12.8-fold and 19.9-fold at 15.6 and 31.3 nM, respectively, compared to 3.2-fold and 9.2-fold for ITF2357 and 2.4-fold and 7.4-fold for PXD101 at 125 and 250 nM, respectively. SAHA increased median p24 levels 1.0-fold, 1.5-fold, and 3.1-fold at 125, 250, and 500 nM, while VPA increased median p24 levels 1.4-fold and 2.8-fold at 0.25 and 0.5 mM (FIG. 3a,b).

In ACH2 cells, with reference to untreated cultures, LBH589 increased median p24 levels 27.7-fold and 51.8-fold at 15.6 and 31.3 nM, respectively, compared to 6.2-fold and 18.5-fold for ITF2357 and 5.8-fold and 18.3-fold for PXD101 at 125 and 250 nM, respectively.

SAHA increased median p24 levels 1.4-fold, 2.4-fold, and 6.9-fold at 125, 250, and 500 nM, while VPA increased median p24 levels 2.1-fold and 7.3-fold at 0.25 and 0.5 mM (FIG. 3c,d).

These differences were statistically significant when comparing LBH589 (15.6 and 31.3 nM) with PXD101 (125 and 250 nM), SAHA (125 and 250 nM), or VPA (0.25 and 0.5 mM) in both ACH2 and U1 cells (P<0.001 for all analyses except P=0.003 for PXD101 in ACH2 cells).

In addition, the increase in p24 with LBH589 at 31.3 nM remained significantly higher than with SAHA at 500 nM (P<0.001).

The direct comparison between LBH589 (15.6 and 31.3 nM) and ITF2357 (125 and 250 nM) showed significantly higher p24 increases with LBH589 treatment in U1 cells, but only a trend towards this in ACH2 cells.

The proportion of primary T-cells expressing the early activation marker CD69 increased in cells treated with increasing concentrations of LBH589 compared to untreated cells with a dose-response relationship (FIG. 4a-h).

The induction of CD69 expression occurred at much lower concentrations of LBH589 than ITF2357, PXD101, and SAHA (FIG. 4i).

Analyses restricted to the memory T-cell subsets showed a similar pattern of increased CD69 expression with increasing HDACi concentration with the highest levels found in LBH589 treated cells (FIG. 4j).

No cytotoxicity above background was observed with LBH589 up to 30 nM.

### Example 2

### The Safety and Efficacy of the Histone Deacetylase Inhibitor Panobinostat for Purging HIV-1 from the Latent Reservoir

Protocol Synopsis

Title

The Safety and Efficacy of The Histone Deacetylase Inhibitor Panobinostat for Purging HIV-1 from The Latent Reservoir (CLEAR) Study.

Problem

Despite effective highly active anti retroviral treatment (HAART), HIV-infection persists and rebounds upon treatment interruption, presumably due to latently infected resting CD4+ T-cells.

To achieve eradication of HIV-infection this reservoir of latently HIV-infected cells must be depleted.

Objectives

Primary Objective

A decrease of >0.5 log₁₀ from baseline to week 16 in the size of the latent HIV+ reservoir as measured by copies of total proviral HIV+DNA per 10⁶ CD4+T+ cells in HIV-infected patients on suppressive HAART.

Secondary Objective

To evaluate the effect of oral Panobinostat on the immunological control of HIV-infection.

To evaluate the safety profile of oral Panobinostat in HIV-infected patients.

To describe the immunological events induced by oral Panobinostat with regard to HIV-specific immunity, T-cell phenotype, immune activation, and cytokine production.

To describe the phylogenetic relationship between episomal HIV-DNA and plasma HIV-RNA induced by oral Panobinostat and how these relate to proviral HIV-DNA and the persistent low-level viremia present prior to study intervention.

To describe genetic, virological, and immunological predictors of treatment response.

Background and Rationale

Early during HIV infection a latent reservoir is established when newly infected CD4+ T-cells revert to a resting memory state.

These cells harbour integrated proviral DNA capable of resuming HIV-expression upon stimulation, but in the inactive state are unrecognizable to the immune system and unresponsive to antiretroviral drugs.

Histone deacetylase (HDAC) inhibitors are a new class of drugs that have been developed for uses in oncology, but which also have the potential to stimulate HIV expression from latently infected resting cells, thereby reducing the viral reservoir.

With HIV-1 expression, the infected cells presumably die due to viral cytopathic effects and/or immune mediated killing and there is a progressive reduction in the size of the reservoir, while concurrent HAART prevents spreading of the infection to new cells.

Panobinostat is a newly developed highly potent HDAC inhibitor that has been administered orally with varying dosing in several clinical trials and was generally well tolerated with no major safety concerns.

In vitro data generated by our group have demonstrated that Panobinostat reverses HIV-latency in latently infected cell lines at sub-toxic concentrations achievable with clinical dosing (see example 1).

Thus, based on example 1 seems Panobinostat to an attractive candidate for purging HIV-1 from the latent viral reservoir.

Hypothesis

Potent HDAC inhibition by oral Panobinostat will purge HIV-1 from latently infected cells and reduce the size of latent HIV-reservoir.

Design

This is a single-group, non-randomized interventional phaseI/II trial designed to evaluate the safety and efficacy of oral Panobinostat in reducing the size of the latent HIV-reservoir in HIV-infected patients on suppressive HAART.

The study will enroll 16 patients.

Each subject will be used as his/her own control in a before-after design:

endpoints measured after study intervention will be compared to baseline for each subject.

Intervention

The investigational drug is defined as Panobinostat, which is a highly potent class I/II HDAC inhibitor belonging to the hydroxamic acid class of compounds.

20 mg Panobinostat will be administered orally on days 1, 3, and 5 (TIW) every other week (QOW) for a total period of 8 weeks.

Subjects will continue background HAART while receiving treatment with the Investigational drug.

The study will comprise three phases:

A pre-treatment screening/observation phase of 4 weeks (weeks 0-4)

A treatment phase of 8 weeks (weeks 4-12), where 20 mg Panobinostat will be administered orally on days 1, 3, and 5 (TIW) every other week (QOW) while maintaining background HAART (co-therapy).

A post-treatment follow-up phase of 24 weeks (weeks 12-36) to evaluate the effect of study treatment, including interruption of HAART from week 24-36.

Methods

HIV-infected patients on stable HAART and plasma HIV-RNA <50 copies/ml for >2 years that fulfil in- and exclusion criteria specified in this protocol will be eligible for the study.

Quantification of proviral HIV-DNA in CD4+ T-cells and resting CD4+ T-cells will be done to evaluate the effect on the size of the latent HIV reservoir.

Episomal HIV-DNA and unspliced HIV-RNA in CD4+ T-cells will be measured to determine induction of HIV-1 expression by the study intervention.

Plasma HIV-RNA and CD4+ cell counts will be measured at selected time points during all three phases of the study.

Plasma HIV-RNA will be measured using standard and ultrasensitive single copy assays (SCA; detection limit 1 copy/ml) to quantify residual viremia.

Peripheral CD4+ counts and isolation of T-cell subsets will be done using fluorescence activated cell sorting (FACS) and negative selection.

Patients will be monitored closely by routine biochemistry, including haematology parameters, ECG, and frequent control visits throughout the study.

Interruption of HAART following the post treatment phase will be performed in weeks 24-36. During HAART interruption, patients will be monitored every two weeks; CD4+ cell-counts <350 cells/mm³ or 2 consecutive viral load measurements >5,000 copies/ml will require resumption of HAART.

Endpoints

Each subject will be used as his/her own control in a before-after design:

endpoints measured after study intervention will be compared to baseline for each subject.

Primary Endpoint

Reduction from pre-treatment phase to week 16 in the size of the latent HIV-reservoir, as measured by copies of total proviral HIV-DNA per 10⁶ CD4+ T-cells.

Secondary Endpoints

Change from pre-treatment phase to week 16 in the size of the latent HIV-reservoir, as measured by copies of integrated proviral HIV-DNA per 10⁶ CD4+ T-cells.

Change from pre-treatment phase to week 16 in the size of the latent HIV-reservoir, as measured by copies of total proviral HIV+DNA per 10⁶ resting CD4+ T-cells.

Proportion of subjects with undetectable proviral HIV-DNA per 10⁶ CD4+ T-cells at week 16.

Change from baseline in unspliced HIV-RNA in CD4+ T-cells during study treatment.

Plasma HIV-RNA, as measured by standard and single copy assay.

Time to viremia >1000 copies/ml during cessation of HAART.

Time to meet criteria to restart HAART.

Safety evaluation, as measured by adverse events (AE), adverse reactions (AR), serious adverse events (SAE), serious adverse reactions (SAR), serious unexpected serious adverse reactions (SUSAR), and dose-limiting toxicity.

Endpoints in Exploratory Analyses

Change from baseline in episomal HIV+DNA in CD4+ T-cells during study treatment.

Numbers and proportions of naive, memory and terminal differentiated T-cells (CD4+ and CD8+).

Numbers and proportions of regulatory T-cells and Th17 cells.

Numbers and proportions of HIV-specific CD4+ and CD8+ T-cells.

HIV specific CD4+ and CD8+ T-cell responses toward selected HIV peptides in stimulatory T-cell assays.

Expression of HIV co-receptors CCR5 and CXCR4, as measured by flow cytometry.

Plasma cytokine and immune activation biomarker levels.

Phylogenetic analyses of circulating and archived HIV strains.

Genetic, virological, and immunological predictors of treatment response.

### Example 3

### Histone Acetylation in PBMC's

Methods

One million freshly isolated PBMC's harvested from CPT vacutainers (BD Bioscience, UK) were washed in ice-cold PBS with 1% FBS.

Subsequently cells were fixed in 200 uL 1% paraformaldehyde for 15 min on ice and afterwards washed in PBS.

Cells were permeabilized in 200 uL 0.1% Triton X-100 for 10 min at RT. Following a wash in FACS buffer (PBS/2% FBS), cells were blocked in PBS with 10% FBS for 20 min at RT. For each patient one sample was incubated for 1 hr at RT with either 5 uL primary anti-acetyl histone H3 antibody (Millipore, DK) or 5μ isotype control (final antibody concentration: 10 μg/m).

Subsequently, cells were washed in FACS buffer and incubated with 5 uL secondary (Alexa-Flour 488 donkey anti-rabbit IgG from Life Technologies, DK) for 1 hr in the dark at RT (final antibody concentration: 6 μg/m). Lastly, cells were washed in FACS buffer, resuspended in PBS and analyzed on a FACS Canto II.

Results

Over the course of panobinostat administration in vivo we determined total cellular histone H3 acetylation levels in eight patients.

A clear and highly significant increase in histone acetylation was observed during panobinostat administration as compared to off-treatment weeks (FIG. 5).

Over the course of weekly dosing (Monday, Wednesday, Friday), the highest levels of histone acetylation was observed on Fridays 8 hrs after dosing.

FIG. 5 shows the relative increase for each patient. On day 4, there was an average increase of 267% from baseline. In the off-treatment weeks, levels of histone acetylation dropped to near baseline levels.

Conclusion

The cyclic dosing schedule pursued here provides a good rationale for viral reactivation strategies. The on/off treatment effect from panobinostat is sustainable over the four consecutive cycles.

### Example 4

### Levels of Cell Associated Unspliced Gag HIV RNA

Methods

RNA purified from 1 million CD4+ T cells was treated with DNAse and split in 4 portions. Preparation of cDNA was performed by Superscript III Reverse Transcriptase as described by the manufacturer using both Oligo dT and random hexamer priming (Life Technologies, DK). Each cDNA sample was assayed in duplicate in a semi-nested real-time PCR quantification yielding eight replicates for each patient sample.

First round amplification was carried out with primers 5′-AACTAGGGAACCCACTGCTTAAG-3′ (SEQ ID NO: 1) and 5′-TCTCCTTCTAGCCTCCGCTAGTC-3′ (SEQ ID NO: 2) with AmpliTaq gold mastermix (Life Technologies, DK) for a limited 15 cycles and input of 5 uL cDNA.

PCR conditions were as follows: A denaturation step at 95° C. for 10 minutes followed by 15 cycles of 20 seconds at 94° C., 40 seconds at 55° C. and 40 seconds at 72° C. Second round real-time PCR was performed using SYBR green mastermix (Life Technologies, DK) and primers 5′-TCTCTAGCAGTGGCGCCCGAACA-3′ (SEQ ID NO: 3) and 5′-TCTCCTTCTAGCCTCCGCTAGTC-3′ (SEQ ID NO: 4) on a Bio-Rad CFX96™ Real-time PCR Platform. PCR conditions were as follows: A denaturation step at 95° C. for 10 minutes followed by 45 cycles of 20 seconds at 94° C., 40 seconds at 55° C., 40 seconds at 72° C. Subsequently, a dissociation curve was obtained by heating from 65° C. to 95° C. with a 0.5° C. increment for 5 seconds. A serial diluted standard allow for relative quantification of HIV Gag RNA levels.

Results

The effect of cyclic panobinostat dosing on the levels of cell-associated unspliced HIV RNA was evident (FIG. 6). In patient A, the levels increased from 178 to 323 and from 57 to 362 Gag HIV RNA copies in the two off/on treatment cycles analyzed. For patient B, the levels were generally higher but a similar increase was observed with off/on treatment status from 504 to 841 and from 892 to 1196 Gag HIV RNA copies in the two cycles analyzed.

Analyzing the four paired on/off points gave a highly significant statistical effect (p=0.008, paired t-test) of panobinostat treatment on cell-associated HIV RNA.

Conclusion

The present inventors provide proof for the ability of Panobinostat to reactivate HIV-1 in vivo as determined by levels of unspliced HIV Gag RNA in CD4+ T cells from panobinostat-treated patients.

### Example 5

### Effect on HIV Proviral DNA in CD4+ T Cells

Methods

DNA was isolated from 1×10⁶ CD4+ T cells using AllPrep DNA/RNA Mini kit according to the manufacturer's specifications (Qiagen, Denmark). Cell equivalents were based on RNAseP amplification. The following primers were used to target human RNAseP 5′-CCCCGTTCTCTGGGAACTC-3′ (forward) (SEQ ID NO: 5) and 5′-TGTATGAGACCACTCTTTCCCATA-3′ (reverse) (SEQ ID NO: 6). Amplification reactions were carried out in duplicates with a primer concentration of 0.5 μM, 10 μL SsoFast™ EvaGreen® Supermix and 1 μL 100-fold diluted DNA as template in 20 μl total volume.

Using the RNAsePgenome copy number, sample DNA was diluted to 50,000 cell equivalents per PCR reaction. To determine the frequency of CD4+ T cells carrying HIV DNA, we prepared a standard curve obtained by serially diluted DNA from 8E5 cells (carrying one integrated provirus per genome) combined with DNA from HIV-negative PBMCs (305, 153, 76, 38, 19, 9 HIV DNA copies and 100,000 HIV negative cell equivalents per well). The following primers were used to target HIV-DNA: 5′-GGTCTCTCTGGTTAGACCAGAT-3′ (forward) (SEQ ID NO: 7) and 5′-CTGCTAGAGATTTTCCACACTG-3′ (reverse) (SEQ ID NO: 8).

The amplification reaction was carried out in triplicates with a primer concentration of 0.5 μM, 10 μL SsoFast™ EvaGreen® Supermix and 100,000 genomes as template in 20 μl total volume on a Bio-Rad CFX96™ Real-time PCR Platform. PCR conditions were as follows: A denaturation step at 95° C. followed by 45 cycles of 10 seconds at 95° C. and 25 seconds at 62° C. Subsequently a melt curve was obtained heating from 65° C. to 95° C. with a 0.5° C. increment for 5 seconds. Cycle cut off was set at 39 cycles. Amplification products were verified by dissociaton curve. Copy number of HIV DNA per 1×106 CD4+ T cells was calculated from the real-time PCR results.

From the HIV DNA standard curve we set a lower limit of quantification of 12 copies HIV DNA/10⁶ CD4+ T cells.

Samples were drawn from two patients at Baseline (prior to initiation of panobinostat treatment) and after 4 weeks and 8 weeks cyclic panobinostat treatment 20 mg TIW every other week.

Results

Over the course of cyclic panobinostat administration in vivo we determined total proviral DNA content in purified CD4+ T cell in two HIV-1 patients. In both patients we observed a drop in proviral DNA content to below the level of detection of the assay as seen in FIG. 7. Patient A had a mean proviral DNA level of 46 copies/10⁶ CD4+ T cell at baseline that within the first two treatment cycles (4 weeks) dropped below detection and remained low at the end of treatment. Patient B had a higher level (173 copies/10⁶ CD4+ T cells) at baseline, which was slightly decreased (125 copies/10⁶ CD4+ T cell) after 4 weeks but became undetectable at the end of treatment. These declines corresponded to a 3.6 and 14.4 fold decline in proviral DNA content in patient A and B, respectively.

Conclusion

The proviral DNA load is a measure of the size of the viral reservoir in HIV-1 patients. The observed drop to below levels of detection in the PCR assay appears to be of major significance with regard to the utility of panobinostat as a prime candidate for HIV-1 eradication purposes.

### Example 6

### Levels of CD4+ T Cells During Panobinostat Treatment

Background

HIV infects CD4+ T cells, which leads to both direct and indirect loss of this T cell subset.

Methods

Measurement of CD4+ T cell counts was performed by flow cytometry as part of routine diagnostics evaluation of HIV-1 patients.

Results

The effect of intermittent panobinostat dosing over 51 days on CD4+ T cell counts in blood is found in FIG. 8. Clearly, there were no immune deterioration in this specific T cell subset.

CD4+ T cells are the key determinant for immunocompetence in HIV infected individuals when stable viral suppressive antiretroviral treatment is maintained.

Conclusion

The TIW oral dosing of 20 mg every other week does not lead to depletion of CD4 T cells nor does it give rise to safety concerns supporting that panobinostat can be safely administered to HIV-infected adults on HAART.

1. A method for inhibiting or treating HIV-1 in an individual comprising selecting an individual for an inhibition or treatment of HIV-1 and providing said individual Panabinostat.

2-20. (canceled)

21. The method of claim 1, wherein said individual is selected for treatment of HIV-1.

22. The method of claim 1, wherein said HIV-1 is a latent HIV-1.

23. The method of claim 1, wherein said Panobinostat is administered in a dose of 5-60 mg.

24. The method of claim 1, wherein said Panobinostat is administered in a dose of 20 mg.

25. The method of claim 22, wherein said Panobinostat is administered in a dose of 5-60 mg.

26. The method of claim 22, wherein said Panobinostat is administered in a dose of 20 mg.

27. The method of claim 1, wherein said Panobinostat is administered in a phase defined by:
phase (i): a dose of Panobinostat on days 1, 3 and 5 of every other week for 8 weeks, while maintaining background HAART as co-therapy.

28. The method of claim 27, further comprising a phase (ii) defined by:
phase (ii): a post-treatment phase of 24 weeks to evaluate the effect of the Panobinostat treatment, where HAART is interrupted the last 8 weeks.

29. The method of claim 28, further comprising a phase (iii) defined by:
phase (iii): repeating (i), and/or (ii).

30. The method of claim 22, wherein said Panobinostat is administered in a phase defined by:
phase (i): a dose of Panobinostat on days 1, 3 and 5 of every other week for 8 weeks, while maintaining background HAART as co-therapy.

31. The method of claim 30, further comprising, a phase (ii) defined by:
phase (ii): a post-treatment phase of 24 weeks to evaluate the effect of the Panobinostat treatment, where HAART is interrupted the last 8 weeks.

32. The method of claim 31, further comprising a phase (iii) defined by:
phase (iii): repeating (i), and/or (ii).

33. The method of claim 27, wherein said Panobinostat is administered in a dose of 5-60 mg.

34. The method of claim 28, wherein said Panobinostat is administered in a dose of 5-60 mg.

35. The method of claim 29, wherein said Panobinostat is administered in a dose of 5-60 mg.

36. The method of claim 30, wherein said Panobinostat is administered in a dose of 5-60 mg.

37. The method of claim 31, wherein said Panobinostat is administered in a dose of 5-60 mg.

38. The method of claim 32, wherein said Panobinostat is administered in a dose of 5-60 mg.

39. A method for depleting a HIV-1 reservoir in an individual comprising selecting an individual for depletion of said HIV-1 reservoir and providing said individual Panabinostat.
